(19) United States
(12) Patent Application Publication
(10) Pub. No.: US 2026/0108756 A1
(43) Pub. Date: Apr. 23, 2026

(54) MOBILE ORAL-CANCER SCREENING AND TREATMENT SYSTEM

(71) Applicant: Arizona Board of Regents on Behalf of the University of Arizona, Tucson, AZ (US)

(72) Inventor: Rongguang Liang, Tucson, AZ (US)

(21) Appl. No.: 19/117,886

(22) PCT Filed: Sep. 28, 2023
(86) PCT No.: PCT/US2023/075391

§ 371 Date: Apr. 2, 2025

Related U.S. Application Data
(60) Provisional application No. 63/378,550, filed on Oct. 6, 2022.

(51) Int. Cl. A61N 5/06; A61B 5/00

(52) U.S. Cl. CPC A61N 5/062; A61B 5/0088; A61B 5/4836; A61N 5/0603; A61N 2005/0606; A61N 2005/063; A61N 2005/0651; A61N 2005/0661; A61N 2005/0665

(57) ABSTRACT

Methods and systems for detection and diagnosis of oral lesions and application of treatment are described. One example mobile intraoral system includes an intraoral probe with a plurality of windows and a first plurality of light sources positioned in its distal section. The intraoral probe also includes one or more additional light sources operable at one or more wavelengths in the range 600 nm to 700 nm, one or more heat sinks, and one or more optical waveguides coupled to the additional light sources. The mobile intraoral system is selectively operable in a first mode of operation for imaging and diagnosis of suspicious regions in the oral cavity, and a second mode of operation for administering photodynamic therapy (PDT). The intraoral probe's distal section is bendable to allow insertion in the oral cavity and targeting a particular location of the oral cavity for administration of the PDT.

### CROSS-REFERENCE TO RELATED APPLICATION(S)

This application claims priority to the provisional application with Ser. No. 63/378,550 titled “MOBILE ORAL-CANCER SCREENING AND TREATMENT SYSTEM,” filed Oct. 6, 2022. The entire contents of the above noted provisional application are incorporated by reference as part of the disclosure of this document.

### STATEMENT REGARDING FEDERALLY SPONSORED RESEARCH

This invention was made with government support under Grant No. CA239682 awarded by National Institutes of Health. The government has certain rights in the invention.

### TECHNICAL FIELD

The technology described in this patent document relates to methods and devices for imaging and monitoring oral regions and administration photodynamic therapy.

### BACKGROUND

Oral and oropharyngeal squamous cell carcinoma (OSCC) together rank as the sixth most common cancer worldwide, with over 640,000 new cases of oral cancer occur worldwide each year. About two-thirds of oral cancers occur in low- and middle-income countries (LMICs), with very high rates in South and South-East Asia (e.g., in Bangladesh, India, Malaysia, Nepal, Pakistan, Sri Lanka. Thailand, and Vietnam). Using India as an example, oral cancer is the most common cancer, accounting for 40% of all cancers overall, and for more than 50% of all cancers in some areas of the country. The Indian sub-continent accounts for one-third of the world's cases. While there exists an urgent need to provide health workers, especially those with limited infrastructure, an effective tool for screening high-risk populations, obtaining simple triage instructions, and diagnosing and monitoring oral lesions, it is also greatly beneficial to provide a cost effective and convenient tool for administering the requisite therapy once the diagnosis has taken place, and to further administer and monitor the required therapy during the course of treatment.

### SUMMARY

The disclosed embodiments, among other features and benefits, relate to compact and low-cost devices and associated methods enable the detection and diagnosis of oral lesions and cancerous growths, and application of treatment using those devices.

One example embodiment relates to a mobile intraoral system that includes an intraoral probe having an elongate body, a distal section including a plurality of windows, and a handle area positioned at a distance from the distal section. The intraoral probe also includes a first plurality of light sources positioned in the distal section of the intraoral probe and configured to provide illumination at a first and a second ranges of wavelengths for illuminating a region in an oral cavity. The intraoral probe also includes one or more additional light sources positioned at or about the handle section, away from the distal section, wherein the one or more additional light sources are operable at one or more wavelengths in the range 600 nm to 700 nm. The intraoral probe further includes one or more heat sinks positioned inside the intraoral probe, proximate to and in contact with the one or more additional light sources to enable dissipation of heat when the one or more additional light sources are operating, and one or more optical waveguides coupled to the one or more additional light sources to deliver light from the one or more additional light sources to one or more windows in the distal section of the intraoral probe. The mobile intraoral system is selectively operable in two modes of operation including a first mode of operation in which the first plurality of light sources is operable to enable imaging and diagnosis of suspicious regions in the oral cavity, and a second mode of operation in which the one or more additional light sources are operable to administer photodynamic therapy (PDT) to one or more areas in the oral cavity. The intraoral probe's distal section is bendable to allow insertion in the oral cavity and targeting a particular location of the oral cavity for administration of the PDT.

### BRIEF DESCRIPTION OF THE DRAWINGS

FIGS. 1A to 1E illustrates an example mobile device and the associated example intraoral probe in accordance with example embodiments.

FIGS. 2A and 2B illustrate diagrams of an intraoral probe in accordance with an example embodiment.

FIG. 3 illustrate another intraoral probe in accordance with one or more example embodiments.

FIG. 4 illustrates another intraoral probe where the LEDs and the associated electronics and heat dissipation mechanisms are included as part of a mobile device in accordance with an example embodiment.

FIG. 5 illustrates another intraoral probe where the LEDs and the associated electronics and heat dissipation mechanisms are included as part of an in-line module in accordance with an example embodiment.

FIG. 6 illustrates diagrams of intraoral probes and the optical channels therein at different cross-sectional areas of the intraoral probe and the associated cable in accordance with example embodiments.

### DETAILED DESCRIPTION

The large number of oral cancers is exacerbated by the relatively low survival rates, especially in those instances where the cancer is not timely diagnosed. For example, the five-year survival rate in the United States for those with localized disease at diagnosis is 83% compared with only 32% for those whose cancer has metastasized to other parts of the body. The survival rate is only 10% to 40% and cure rates around 30% in developing countries. The poor survival rate is mainly due to late diagnosis and the resultant progression of disease to an advanced stage at diagnosis. The patients are often referred to a specialist with advanced stage disease when the cancer has already spread from the oral cavity to the neck and distant sites. The reasons for delay in diagnosis of Oral and oropharvngeal squamous cell carcinoma (OSCC) are complex and influenced by the patient, providers, infrastructure and health systems. Diagnosis is often delayed in marginalized, at-risk populations living in rural areas with lack of access to primary health care, low health literacy, and poor adherence to referral and follow up. In rural and resource-restricted settings, there are few primary healthcare providers and even fewer specialists. Early malignant changes are often subtle, with visible symptoms occurring relatively late in the process. An improvement in our ability to detect premalignant and early malignant changes would improve overall survival. It is also greatly beneficial, once the cancer is diagnosed, to provide a cost effective method using a convenient tool for administering the requisite therapy, and to further administer and monitor the required therapy during the course of treatment.

The disclosed embodiments, among other features and benefits, relate to compact and low-cost devices and associated methods that not only enable the detection and diagnosis of oral lesions and cancerous growths, but to also apply treatment using the same device.

One method for treating oral cancers is photodynamic therapy (PDT) which uses light at a particular wavelength and dose to attack cancerous legions. PDT operates by relying on accumulation of a photosensitive dye in premalignant and malignant lesions; when such regions are illuminated with the light for a certain period of time, a photochemical reaction between the sensitizer and light will occur, which subsequently can damage the cancerous cells. Traditionally, a wavelength of about 630 nm has been considered a suitable wavelength for PDT.

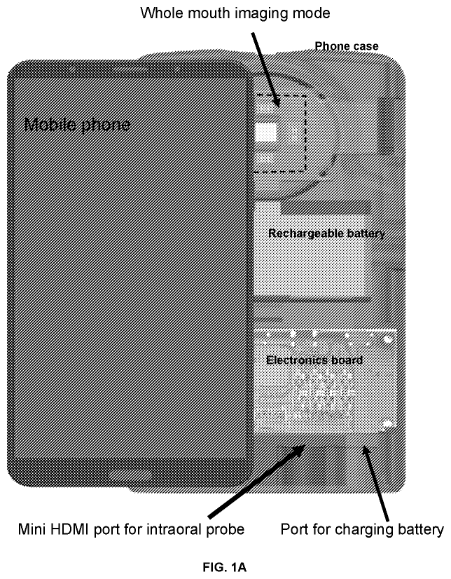

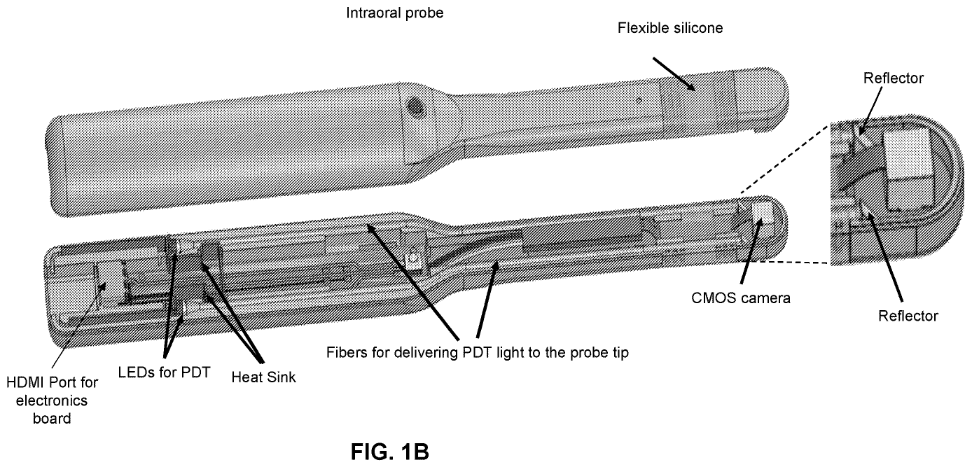

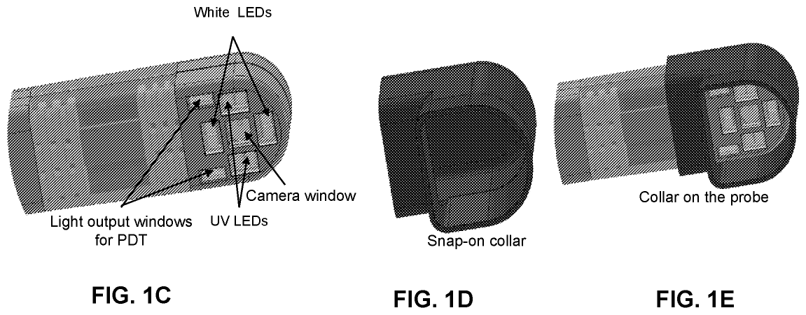

FIG. 1A illustrates an example mobile device, and FIGS. 1B to 1E illustrate an example intraoral probe in accordance with example embodiments. In FIG. 1A, the mobile device's camera can be used for taking images of the whole mouth, and the mobile device may be augmented to include additional light sources (if needed) to illuminate the mouth. The electronics board in the mobile device can be used to control the various operations of the intraoral probe as will be discussed in the sections that follow. The intraoral probe may be connected to the mobile device via wired or wireless connections. For example, in some embodiments, the mini HDMI port of the mobile device can be used for connecting the intraoral probe.

FIG. 1B illustrates an intraoral probe in accordance with an example embodiment. As shown in the bottom panel, two LEDs are positioned in the base section of the intraoral probe to produce light for PDT. Light from the LEDs is provided to the tip of the intraoral probe via optical waveguides (e.g., fibers). One or more heat sinks are also provided to assist with dissipation of heat that is generated by the LEDs. Positioning of the LEDs far away from the tip allows the tip to remain at a reasonably low temperature, which enables the tip of the intraoral probe to be remain in patient's mouth for the duration of therapy. While in some embodiments one LED may be used, inclusion of two LEDs provides for better heat management while allowing higher intensity light to be provided for treatment. In some embodiments more than two LEDs may be used.

In an example configuration, LEDs are two high power red LEDs (635 nm) that are mounted in 2 mm thick aluminum LED boards in the handle of the probe. The optical waveguides are two 1.5 mm diameter multimode fibers that deliver the light to the distal end of the intraoral probe. Two reflectors at the distal end, as shown in FIG. 1B (magnified inset), can direct the light from the fibers to outside of the intraoral probed in the direction of the tissue surfaces. The electronics board of the mobile device is designed to include three outputs for white light, UV light, and red light LEDs. The mobile device can be further augmented to include a rechargeable 7.4V/2S 1000 mAh 20C LiPO battery to ensure sufficient current generation for the operation of red LEDs for PDT. In another example configuration, one or more red laser diodes are used for PDT.

The tip of an example intraoral probe is shown in FIG. 1C, which includes two while LEDs, two UV LEDs and a camera window that assist with imaging and diagnosis of cancerous and pre-cancerous lesions. The UV LEDs are also optimal to excite ALA-induced PpIX fluorescence. Further details regarding the use of light sources for imaging and diagnosis are described in PCT Publication WO 2021/207079 A1, also published as U.S. Patent Application No. US2023/0148852A1. Referring back to FIG. 1C, the tip of the intraoral probe further includes two windows that allows PDT light from the LEDs to reach outside of the probe and directed to the target region. FIG. 1D illustrates an example snap-fit collar for the intraoral probe, and FIG. 1E illustrates the collar when positioned on the tip of the intraoral. The collar prevents background light interference during the imaging process, and also prevents other areas of the oral cavity from receiving light during administration of PDT to a particular region of the oral cavity.

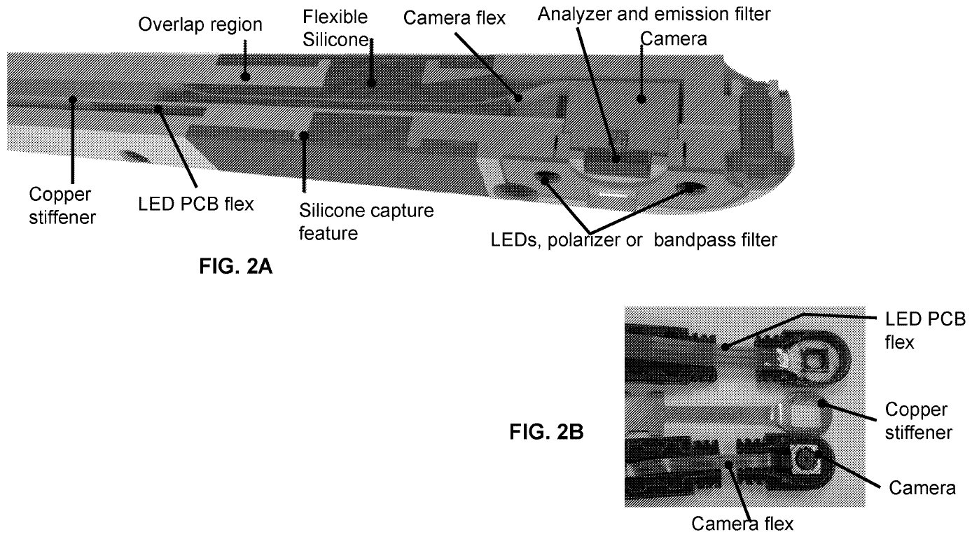

FIG. 2A illustrates an example diagram of atop section of an intraoral probe, showing a polarizer, bandpass filter, analyzer and emission filter positioned at the tip of the probe. The polarizer positioned in front of each white LED allows polarized illumination to be provided to the target region; another polarizer in front of the imaging optics can help with removal of specular reflection. To capture autofluorescence images, a longpass filter, which only passes the light with wavelength longer than 450 nm, can be placed in front of the imaging optics to block the illumination light from UV LEDs with center wavelength of 405 nm (a shortpass filter is placed in front of each UV LED to reduce the excitation light leakage). Further details are described in PCT Publication WO 2021/207079 A1, also published as U.S. Patent Application No. US2023/0148852A1. FIG. 2A also illustrates the flexible silicone sections, flexible PCB flex and other components.

FIG. 2B illustrates the camera, copper stiffener and LED PCB and camera flex connectors that can be used to provide connectivity and to provide structural reinforcement. To obtain sufficient resolution over the large field of view in the oral cavity, a low-cost, high performance 5-megapixel OV5648 CMOS sensor with 2592×1944 pixels (OmniVision Technologies Corp) can be used. The imaging optics can be mounted in a voice coil actuator for autofocus, which will enable the users to capture good image over a relatively large range of focusing distances.

The below provides component characteristics and performance metrics for an example intraoral diagnosis and treatment in accordance with an example embodiment.

| SYSTEM | | | |
|---|---|---|---|
| PARAMETERS | AFI | pWLI | PDT |
| Light source | UV LED | White LED | Red LED |
| Detector | CMOS Sensor | CMOS Sensor | N/A |
| Light Source Spectrum | 405 | 450-650 | 625-650 |
| (nm) | | | |
| Detection Spectrum (nm) | 465-650 | 450-650 | N/A |
| Working Distance (nm) | 10-40 | 10-40 | N/A |
| Field of View (mm) | 30 × 20 at 25 mm | 30 × 20 at 25 mm | 30 × 20 at 25 mm |
| | working | working distance | working distance |
| | distance | | |
| Lateral Resolution (micron) | 25 | 25 | N/A |
| Light Intensity (mW/cm²) | Tunable | Tunable | >30 |

One important system characterization is the energy delivered to the tissue surface for PDT. In some embodiments, the intensity of the light delivered is greater than 30 mW/cm². In other embodiments the intensity is greater than 20 mW/cm². The uniformity is better than 80%. In some embodiments, the spectral range for the red LED or red laser diode can be within 600-700 nm.

The disclosed system also includes a mobile application that can include four modules: data input module to record patient information, imaging module to take images from intraoral probe, PDT module, and mobile image classification and segmentation module.

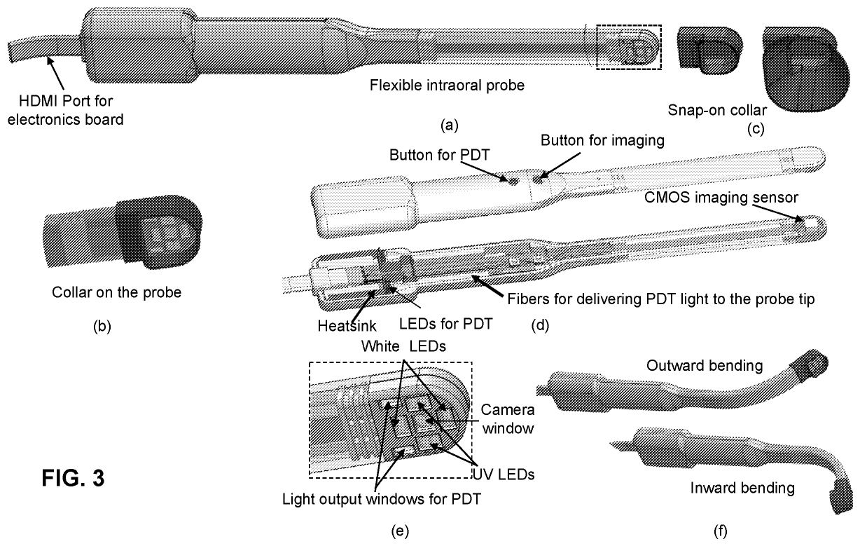

FIG. 3 illustrate an intraoral probe in accordance with some example embodiments. Some of the probe's components are similar to those depicted in the earlier figures. The noted additions are the depiction of the full probe in a closed condition (panel (a)), the capability to include multiple snap-on collars (panel (c)), addition of a separate on/off switch for PDT (panel (d)) and inward and outward bending capabilities (panel (f)). For instance, panel (c) illustrates two collars with differing end sections that allow a smaller or a larger section of the oral cavity to be targeted (for imaging and for PDT administration).

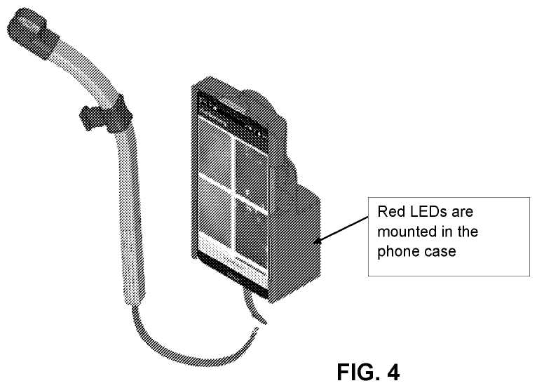

FIG. 4 illustrates an alternate embodiment of an intraoral probe where the LEDs for PDT and the associated electronics and heat dissipation mechanisms are included as part of the mobile device—for example, as an additional compartment that can be mounted on a mobile phone. Light from the red LEDs is delivered via an optical waveguide (e.g., optical fibers) that is tethered to the interaural probe. The light can be delivered to the distal end via internal optical waveguides as illustrated earlier. The configuration of FIG. 4 simplifies the design of heat sinks, and eliminates any heat dissipation considerations in the probe itself. In some embodiments some or all of the other light sources (the UV and/or the white light source) can be included in the compartment (e.g., the “lightbox”). In this embodiment, red laser diodes, instead of red LEDs, can be used.

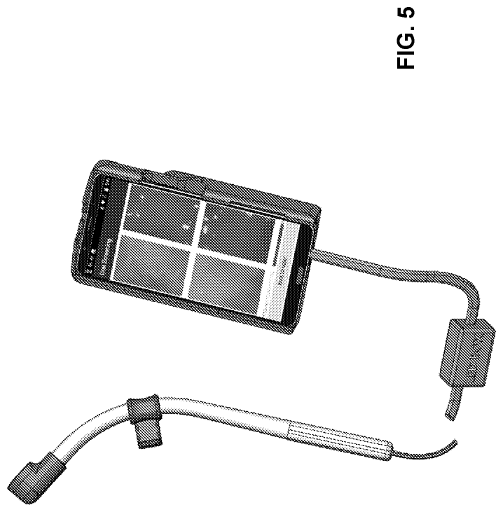

FIG. 5 illustrates another alternate embodiment of an intraoral probe where the LEDs for PDT and the associated electronics and heat dissipation mechanisms are included as part of an in-line module between the mobile device and the intraoral probe. The configuration of FIG. 5 does not change the physical characteristics (e.g., weight and size) of the mobile device while still providing heat dissipation management benefits similar to those in FIG. 4.

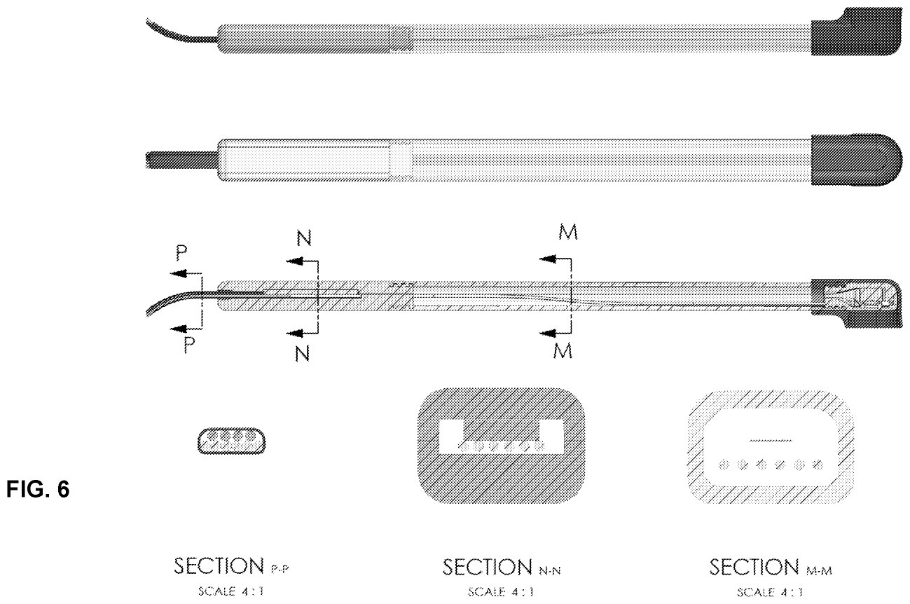

FIG. 6 illustrates example diagrams of the intraoral probes and the optical channels therein at different cross-sectional areas of the intraoral probe and the associated cable. The P-P cross-sectional view illustrates six fibers (shown with lighter color) that are used to deliver the light and 4 cables (shown with a darker color) that are used to control the camera; the N-N cross-section view illustrates the six optical channels that are secured in place in the handle area of the intraoral probe; the M-M cross-sectional view shows the optical channels in the middle section of the intraoral probe.

It is understood that the various disclosed embodiments may be implemented individually, or collectively, in devices comprised of various optical components, electronics hardware and/or software modules and components. These devices, for example, may comprise a processor, a memory unit, an interface that are communicatively connected to each other, and may range from desktop and/or laptop computers, to mobile devices and the like. The processor and/or controller can perform various disclosed operations based on execution of program code that is stored on a storage medium. The processor and/or controller can, for example, be in communication with at least one memory and with at least one communication unit that enables the exchange of data and information, directly or indirectly, through the communication link with other entities, devices and networks. The communication unit may provide wired and/or wireless communication capabilities in accordance with one or more communication protocols, and therefore it may comprise the proper transmitter/receiver antennas, circuitry and ports, as well as the encoding/decoding capabilities that may be necessary for proper transmission and/or reception of data and other information. For example, the processor may be configured to receive electrical signals or information from the disclosed sensors (e.g., CMOS sensors), and to process the received information to produce images or other information of interest. The communications between the disclosed detectors and the processors may be carried out via near field communication (NFC) protocols.

The processing devices that are described in connection with the disclosed embodiments can be implemented as hardware, software, or combinations thereof. For example, a hardware implementation can include discrete analog and/or digital components that are, for example, integrated as part of a printed circuit board. Alternatively, or additionally, the disclosed components or modules can be implemented as an Application Specific Integrated Circuit (ASIC) and/or as a Field Programmable Gate Array (FPGA) device. Some implementations may additionally or alternatively include a digital signal processor (DSP) that is a specialized microprocessor with an architecture optimized for the operational needs of digital signal processing associated with the disclosed functionalities of this application.

One aspect of the disclosed embodiments relates to a mobile intraoral system that includes an intraoral probe comprising an elongate body, a distal section including a plurality of windows, and a handle area positioned at a distance from the distal section. The mobile intraoral system further comprises a first plurality of light sources positioned in the distal section of the intraoral probe and configured to provide illumination at a first and a second ranges of wavelengths for illuminating a region in an oral cavity, and one or more additional light sources positioned at or about the handle section, away from the distal section, wherein the one or more additional light sources is operable at one or more wavelengths in the range 600 nm to 700 nm. The mobile intraoral system additionally includes one or more heat sinks positioned inside the intraoral probe, proximate to and in contact with the one or more additional light sources to enable dissipation of heat when the one or more additional light sources are operating, and one or more optical waveguides coupled to the one or more additional light sources to deliver light from the one or more additional light sources to one or more windows in the distal section of the intraoral probe. The mobile intraoral system is selectively operable in two modes of operation including a first mode of operation in which the first plurality of light sources is operable to enable imaging and diagnosis of suspicious regions in the oral cavity, and a second mode of operation in which the one or more additional light sources are operable to administer photodynamic therapy (PDT) to one or more areas in the oral cavity. The intraoral probe's distal section is bendable to allow insertion in the oral cavity and targeting a particular location of the oral cavity for administration of the PDT.

In one example embodiment, the mobile intraoral system further includes one or more collars that are attachable to the distal section. The one or more collars include an outwardly flared end section that allows light from the one or more additional light sources to be directed to the particular location of the oral cavity while reducing or eliminating leakage to other sections of the oral cavity. In another example embodiment, the mobile intraoral system includes multiple collars, wherein each collar differs from another collar in a size of the outwardly flared end section. In yet another example embodiment, each of the one or more collars is configured to be snap-fitted onto the distal section. In still another example embodiment, the mobile intraoral system further includes one or more reflectors positioned in the distal section of the intraoral probe to receive light from the one or more additional light sources through the one or more optical waveguides and to reflect the received light in the direction of the one or more windows in the distal section of the intraoral probe.

According to another example embodiment, the one or more optical waveguides include optical fibers. In another example embodiment, the one or more additional light sources are configured to produce a light intensity of at least 20 mW/cm². In still another example embodiment, the mobile intraoral system includes two separate on-off switches, wherein a first on-off switch is configured to control the operation of the mobile intraoral system in the first mode of operation, and a second on-off switch is configured to control the operation of the mobile intraoral system in the second mode of operation. In yet another example embodiment, the first plurality of light sources includes a light source operable in an ultraviolet region of wavelengths, and a light source operable to produce white light.

In one example embodiment, the mobile intraoral system includes a sensor positioned in the distal section and configured to receive light from the suspicious regions in the oral cavity in response to illumination by light from the first plurality of light sources, wherein the mobile intraoral system includes a mobile electronic device communicatively coupled to the intraoral probe and configured to provide one or more of the following: control the first plurality of light sources, control the one or more additional light sources, or receive electrical signals from the sensor corresponding to the light received from the suspicious regions in the oral cavity. In another example embodiment, the mobile intraoral system includes a mobile application implemented as part of the mobile electronic device, wherein the mobile application comprises a plurality of modules, including a data input module to record patient information, an imaging module to receive information associated with images produced by the intraoral probe, and a PDT module configured to control administration of the PDT.

In yet another example embodiment, the PDT module is configured to enable administration of the PDT for a particular duration of time and at a particular dosage level. In another example embodiment, the mobile electronic device is connected to the intraoral probe by an electrical cable. According to another example embodiment, the one or more additional light sources include on or more light emitting diodes (LEDs) or one of more lasers.

Another aspect of the disclosed embodiments relates to a mobile intraoral system that includes an intraoral probe comprising an elongate body, a distal section including a plurality of windows, and a handle area positioned at a distance from the distal section. The mobile intraoral system further includes a mobile electronic device communicatively coupled to the intraoral probe, a compartment including one or more light sources operable at one or more wavelengths in the range 600 nm to 700 nm, where the compartment is a separate component from the intraoral probe and includes one or more heat sinks proximate to and in contact with the one or more light sources to enable dissipation of heat when the one or more light sources are operating. The mobile intraoral system also includes an optical cable connecting the compartment to the intraoral probe and including one or more optical fibers to enable delivery of light from the compartment to the intraoral probe. The mobile intraoral system additionally includes one or more optical waveguides inside the intraoral probe configured to deliver light received from the optical cable to one or more windows in the distal section of the intraoral probe. The intraoral probe's distal section is bendable to allow insertion in the oral cavity and targeting a particular location of the oral cavity for administration of photodynamic therapy (PDT).

In one example embodiment, the compartment is mounted on the mobile electronic device of the mobile intraoral system. In another example embodiment, the compartment is a separate component from the mobile electronic device, and the mobile intraoral system includes a link to the compartment for enabling communications with components of the compartment. According to another example embodiment, the compartment further includes a light source operable in an ultraviolet region of wavelengths, and a light source operable to produce white light. In yet another example embodiment, the intraoral probe further includes, in the distal section, a light source operable in an ultraviolet region of wavelengths, and a light source operable to produce white light. In still another example embodiment, the mobile intraoral system includes a sensor positioned in the distal section and configured to receive light from one or more regions in the oral cavity in response to illumination by light from the distal section of the intraoral probe.

In another example embodiment, the mobile electronic device includes a processor and a memory including instructions stored thereon, wherein the instructions upon execution by the processor cause the processor to trigger the operation of one or more light sources to administer the PDT for a particular duration of time and at a particular dosage level. In one example, embodiment, the one or more light sources include on or more light emitting diodes (LEDs) or one of more lasers.

Various information and data processing operations described herein may be implemented in one embodiment by a computer program product, embodied in a computer-readable medium, including computer-executable instructions, such as program code, executed by computers in networked environments. A computer-readable medium may include removable and non-removable storage devices including, but not limited to, Read Only Memory (ROM), Random Access Memory (RAM), compact discs (CDs), digital versatile discs (DVD), etc. Therefore, the computer-readable media that is described in the present application comprises non-transitory storage media. Generally, program modules may include routines, programs, objects, components, data structures, etc. that perform particular tasks or implement particular abstract data types. Computer-executable instructions, associated data structures, and program modules represent examples of program code for executing steps of the methods disclosed herein. The particular sequence of such executable instructions or associated data structures represents examples of corresponding acts for implementing the functions described in such steps or processes.

The foregoing description of embodiments has been presented for purposes of illustration and description. The foregoing description is not intended to be exhaustive or to limit embodiments of the present invention to the precise form disclosed, and modifications and variations are possible in light of the above teachings or may be acquired from practice of various embodiments. The embodiments discussed herein were chosen and described in order to explain the principles and the nature of various embodiments and its practical application to enable one skilled in the art to utilize the present invention in various embodiments and with various modifications as are suited to the particular use contemplated. While operations are depicted in the drawings in a particular order, this should not be understood as requiring that such operations be performed in the particular order shown or in sequential order, or that all illustrated operations be performed, to achieve desirable results. The features of the embodiments described herein may be combined in all possible combinations of methods, apparatus, modules, and systems.

1. A mobile intraoral system, comprising:
an intraoral probe comprising an elongate body, a distal section including a plurality of windows, and a handle area positioned at a distance from the distal section;
a first plurality of light sources positioned in the distal section of the intraoral probe and configured to provide illumination at a first and a second ranges of wavelengths for illuminating a region in an oral cavity;
one or more additional light sources positioned at or about the handle section, away from the distal section, the one or more additional light sources operable at one or more wavelengths in the range 600 nm to 700 nm;
one or more heat sinks positioned inside the intraoral probe, proximate to and in contact with the one or more additional light sources to enable dissipation of heat when the one or more additional light sources are operating; and
one or more optical waveguides coupled to the one or more additional light sources to deliver light from the one or more additional light sources to the plurality of windows in the distal section of the intraoral probe,
wherein the mobile intraoral system is selectively operable in two modes of operation including a first mode of operation in which the first plurality of light sources is operable to enable imaging and diagnosis of suspicious regions in the oral cavity, and a second mode of operation in which the one or more additional light sources are operable to administer photodynamic therapy (PDT) to one or more areas in the oral cavity, and
wherein the intraoral probe's distal section is bendable to allow insertion in the oral cavity and targeting a particular location of the oral cavity for administration of the PDT.

2. The mobile intraoral system of claim 1, further including:
one or more collars that are attachable to the distal section, the one or more collars including an outwardly flared end section that allows light from the one or more additional light sources to be directed to the particular location of the oral cavity while reducing or eliminating leakage to other sections of the oral cavity.

3. The mobile intraoral system of claim 2, including multiple collars, wherein each collar differs from another collar in a size of the outwardly flared end section.

4. The mobile intraoral system of claim 2, wherein each of the one or more collars is configured to be snap-fitted onto the distal section.

5. The mobile intraoral system of claim 1, further including one or more reflectors positioned in the distal section of the intraoral probe to receive light from the one or more additional light sources through the one or more optical waveguides and to reflect the received light in the direction of the one or more windows in the distal section of the intraoral probe.

6. The mobile intraoral system of claim 1, wherein the one or more optical waveguides include optical fibers.

7. The mobile intraoral system of claim 1, wherein the one or more additional light sources are configured to produce a light intensity of at least 20 mW/cm².

8. The mobile intraoral system of claim 1, including two separate on-off switches, wherein a first on-off switch is configured to control the operation of the mobile intraoral system in the first mode of operation, and a second on-off switch is configured to control the operation of the mobile intraoral system in the second mode of operation.

9. The mobile intraoral system of claim 1, wherein the first plurality of light sources includes a light source operable in an ultraviolet region of wavelengths, and a light source operable to produce white light.

10. The mobile intraoral system of claim 1, comparing a sensor positioned in the distal section and configured to receive light from the suspicious regions in the oral cavity in response to illumination by light from the first plurality of light sources, wherein the mobile intraoral device comprises a mobile electronic system communicatively coupled to the intraoral probe and configured to provide one or more of the following:
control the first plurality of light sources,
control the one or more additional light sources, or
receive electrical signal from the sensor corresponding to the light received from the suspicious regions in the oral cavity.

11. The mobile intraoral system of claim 10, including a mobile application implemented as part of the mobile electronic device, wherein the mobile application comprises a plurality of modules, including a data input module to record patient information, an imaging module to receive information associated with images produced by the intraoral probe, and a PDT module configured to control administration of the PDT.

12. The mobile intraoral system of claim 11, wherein the PDT module is configured to enable administration of PDT for a particular duration of time and at a particular dosage level.

13. The mobile intraoral system of claim 10, wherein the mobile electronic device is connected to the intraoral probe by an electrical cable.

14. The mobile intraoral system of claim 1, wherein the one or more additional light sources include on or more light emitting diodes (LEDs) or one of more lasers.

15. A mobile intraoral system, comprising:
an intraoral probe comprising an elongate body, a distal section including a plurality of windows, and a handle area positioned at a distance from the distal section;
a mobile electronic device communicatively coupled to the intraoral probe;
a compartment including one or more light sources operable at one or more wavelengths in the range 600 nm to 700 nm, the compartment being a separate component from the intraoral probe and including one or more heat sinks proximate to and in contact with the one or more light sources to enable dissipation of heat when the one or more light sources are operating;
an optical cable connecting the compartment to the intraoral probe and including one or more optical fibers to enable delivery of light from the compartment to the intraoral probe; and
one or more optical waveguides inside the intraoral probe configured to deliver light received from the optical cable to one or more windows in the distal section of the intraoral probe,
wherein the intraoral probe's distal section is bendable to allow insertion in the oral cavity and targeting a particular location of the oral cavity for administration of photodynamic therapy (PDT).

16. The mobile intraoral system of claim 15, wherein the compartment is mounted on the mobile electronic device of the mobile intraoral system.

17. The mobile intraoral system of claim 15, wherein the compartment is a separate component from the mobile electronic device, and the mobile intraoral system includes a link to the compartment for enabling communications with components of the compartment.

18. The mobile intraoral system of claim 16, wherein the compartment further includes a light source operable in an ultraviolet region of wavelengths, and a light source operable to produce white light.

19. The mobile intraoral system of claim 15, wherein the intraoral probe further includes, in the distal section, a light source operable in an ultraviolet region of wavelengths, and a light source operable to produce white light.

20. The mobile intraoral system of claim 15, comprising a sensor positioned in the distal section and configured to receive light from one or more regions in the oral cavity in response to illumination by light from the distal section of the intraoral probe.

21. The mobile intraoral system of claim 15, wherein the mobile electronic device includes a processor and a memory including instructions stored thereon, wherein the instructions upon execution by the processor cause the processor to trigger the operation of one or more light sources to administer PDT for a particular duration of time and at a particular dosage level.

22. The mobile intraoral system of claim 15, wherein the one or more light sources include on or more light emitting diodes (LEDs) or one of more lasers.
